(19) United States
(12) Patent Application Publication
(10) Pub. No.: US 2003/0219469 A1
(43) Pub. Date: Nov. 27, 2003

(54) Environmental control device for tissue treatment

(75) Inventors: Royce Johnson, Universal City, TX (US); Jennifer Morgan Johnson, Universal City, TX (US); Jonathan Kagan, Hopkins, MN (US)

Correspondence Address: Kinetic Concepts, Inc., P.O. Box 659508, San Antonio, TX, 78265-9508, US

(73) Assignee: KCI Licensing, Inc., San Antonio, TX (US)

(21) Appl. No.: 10/361,341

(22) Filed: Feb. 10, 2003

Related U.S. Application Data
(60) Provisional application No. 60/355,946, filed on Feb. 11, 2002.

(51) Int. Cl. A61L 15/00

(52) U.S. Cl. 424/445

(57) ABSTRACT

The present invention is an environmental control device for treating tissue, consisting of a chamber having a bellows configuration that is sealable to tissue around its periphery. A resealable access port is provided in order to introduce and extract fluid from the chamber. A lift tab is provided to lift the portal to an angle roughly perpendicular to the tissue, in order to minimize the risk of injury to both the patient and the caregiver.

### RELATED APPLICATIONS

This application claims priority to U.S. provisional patent application No. 60/355,946.

### FIELD OF THE INVENTION

The invention relates to an environmental control device for the treatment of tissue in mammals. More particularly, the invention relates to a tissue treatment system for wounds, burns, skin diseases, gene therapy, and topical treatment of tissue in a closed, localized, and controlled environment.

### BACKGROUND OF THE INVENTION

Treatment of open wounds has been accomplished in both a moist and dry environment. Wounds treated in a moist environment have been shown to accelerate the healing process. Additionally, it would be useful to be able to provide a means for maintaining a treatment in solution within a tissue region for an extended period of time.

The concept of using fluid to treat the wound is generally known. Apparatuses for use in fluid treatment of wounds are also known in the prior art. For example, the system disclosed in U.S. Pat. No. 5,152,757 issued to Eriksson describes a chamber formed by a flexible sheet material having a peripheral adhesive surface. A portal is also described for introduction of treatment fluids within the chamber.

Other systems, including that described in U.S. Pat. No. 3,026,874 issued to Stevens, are comprised of an annular dam that forms a seal around a wound, with a transparent and flexible “window” overlying and secured to the seal, as well as an inlet adapted for connection to a medication source and an outlet port adapted for connection to a vacuum source.

However, the prior art does not address significant risks to the patient and caregiver when injecting fluids into the chamber.

### SUMMARY OF THE INVENTION

An environmental control device for the treatment of tissue according to the present invention comprises a chamber having a bellows configuration that is scalable to tissue around its periphery. A resealable access port is provided in order to introduce and extract fluid from the chamber. The access port includes a lift tab for lifting the portal to an angle roughly perpendicular to the tissue, in order to minimize the risk of injury to both the patient and the caregiver. The chamber is comprised of a transparent layer for viewing the tissue overlaid by the device.

The device serves to provide a controlled environment for tissue treatment. Treatment of wounds can be accomplished by providing a wet environment over the wound site. The chamber is placed over the wound site, and a solution may be injected into the chamber through the injection port. Other elements, such as growth factors or genetically altered cells may be provided within the solution. A simple saline solution may be provided to keep the wound area wet, which has been shown to accelerate wound healing over a dry environment.

An additional embodiment provides for maintenance of growth factors, cytokines, and other genetically altered cells for enhancing wound healing. It is anticipated that cellular matrices, such as that described in U.S. Pat. No. 5,824,331, issued to Usala, and assigned to Encelle, Inc., may be suspended within a gel-like medium, and injected or held within the present invention, in order to facilitate the healing of wounds.

The foregoing has outlined some of the more pertinent objects of the present invention. These objects should be construed to be merely illustrative of some of the more prominent features and applications of the invention. Many other beneficial results can be attained by applying the disclosed invention in a different manner or by modifying the invention as will be described. Accordingly, other objects and a fuller understanding of the invention may be had by referring to the following Detailed Description of the Invention, which includes the preferred embodiment.

### BRIEF DESCRIPTION OF THE DRAWINGS

These and other features and advantages of the invention will now be described with reference to the drawings of certain preferred embodiments, which are intended to illustrate and not to limit the invention, and wherein like reference numbers refer to like components, and in which:

FIG. 1 is an overhead view of an environmental control device for tissue treatment having a needle insertion port utilized in accordance with the present invention.

FIG. 2 is an exploded view of the component parts of an environmental control device for tissue treatment utilized in accordance with the present invention.

FIG. 3 is a cross-sectional view an environmental control device for tissue treatment utilized in accordance with the present invention.

FIG. 4 is a perspective view of an access port and finger tab of an environmental control device for tissue treatment utilized in accordance with the present invention.

FIG. 5 is side angle view of an environmental control device utilized in accordance with the present invention.

FIG. 6 is an overhead view of an alternate embodiment of an environmental control device utilized in accordance with the present invention.

### DESCRIPTION

Although those of ordinary skill in the art will readily recognize many alternative embodiments, especially in light of the illustrations provided herein, this detailed description is exemplary of the preferred embodiment of the present invention, the scope of which is limited only by the claims that may be drawn hereto.

The present invention is an environmental control device for treatment of tissue. The treatment of tissue may include treatment of wounds, including burns, incisional wounds, excisional wounds, ulcers, traumatic wounds, and chronic open wounds. Additionally, tissue treatment may include treatment of skin disorders, hair follicle treatment or stimulation, skin grafting, and introduction of genetically altered or synthetically developed cells or matrices. The term “wet environment” as used herein refers to fluids (in both liquid and/or gaseous form) or gel-like solutions or suspensions. The above referenced treatment modalities and definitions are meant to be exemplary and are not intended to limit the scope of the present invention.

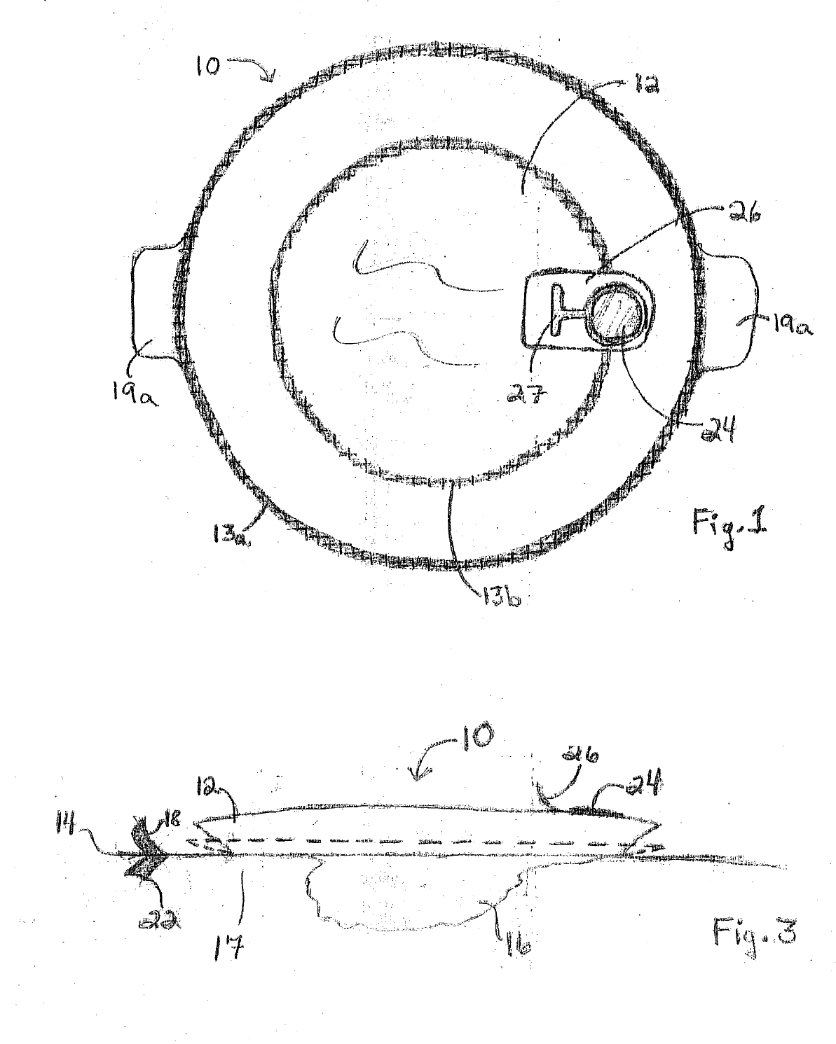

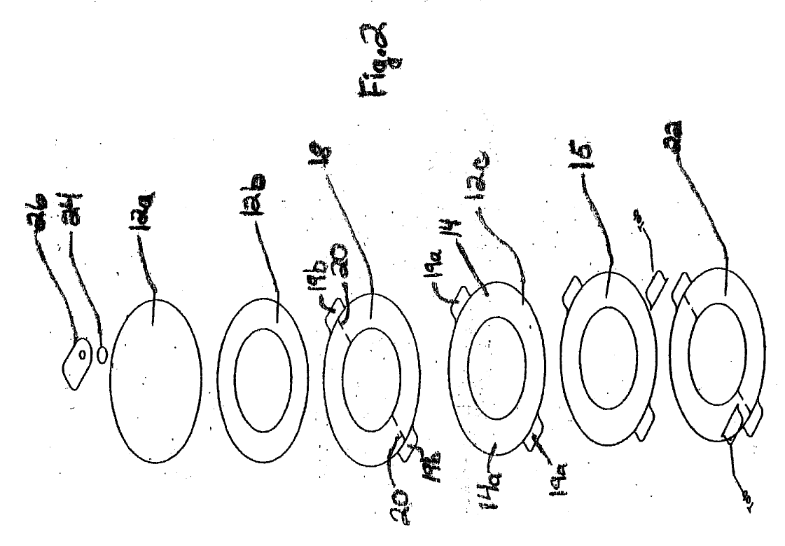

Referring now to FIGS. 1 and 2 in particular, there is illustrated the primary components of a device that operates in accordance with the present invention. The present invention is a device 10 that includes an expandable liquid impermeable membrane 12 that may be pleated or otherwise formed into a bellows chamber.

The bellows membrane 12 may be formed by sealing, such as by a weld, the outer perimeter of a flat sheet of membrane 12a to the outer perimeter of a first annular membrane 12b, which may be ring shaped, to form an outer bond 13a. The inner perimeter of the first annular membrane 12b is fused to the inner perimeter of a similarly shaped second annular membrane 12c to form an inner bond 13b. The second annular membrane 12c serves as an attachment ring 14 of the bellows membrane 12 to provide a surface for attachment of the bellows membrane 12 to tissue 17. Positioning tabs 19a are provided at convenient positions, such as at opposing ends of the attachment ring 14, to allow for ease of manipulation of the bellows membrane 12 during attachment to the tissue 17. The bellows shaped membrane 12 is preferably made of a transparent polyurethane material, in order to create a liquid impermeable seal, while allowing visual inspection of the tissue covered by the device 10.

An adhesive 15 is bonded to the attachment ring 14 to allow the device 10 to be adhered to an area of tissue 17 that requires a controlled environment, such as an open wound 16. The adhesive 15 is comprised of a material that allows it to be easily removable from a laminate, such as the release liner 22, but that also provides for a fluid tight seal to the tissue 17, for an extended period of time. The adhesive 15 is such that it will maintain a fluid tight seal between the attachment ring 14 and the tissue 17, despite the shear and pressure forces that may be applied to the area of the tissue 17 being treated by the device 10 during everyday activities. For example, the adhesive material must be able to maintain a fluid tight seal should the present invention 10 be applied to tissue located on the abdominal region of a patient who is still able to maintain normal daily activities. It is contemplated that an acrylate adhesive may be used, such as that sold by Entrotek Corporation under part number ECA-134. Alternate adhesives may also include polyisobutylene, such as that sold by Corium under the Cor-tak X trademark designation. Silicone adhesives, such as that sold by Nusil Technologies under part number Med 1356, may also be used. Similarly, a styrene adhesive, such as that sold by National Starch Corporation under part number Durotak-87-6173 may also be utilized. The above referenced adhesives are provided by way of example and are not intended to limit the type of adhesive utilized in the present invention. It is to be understood that other adhesives may be utilized that are capable of maintaining a fluid tight seal between the attachment ring 14 and the tissue 17 for up to approximately five to seven days, or longer if necessary, without the necessity of reapplying the adhesive 15 to the device 10.

A stabilizer layer 18 is provided for support of the bellows membrane 12, and is removably attached along the upper portion 14a of the attachment ring 14 prior to adhesion to the tissue 17. The stabilizer layer 18 is a semi-rigid piece of material, such as a stiff paper or thin cardboard material, having a shape and size substantially similar to the attachment ring 14. The stabilizer layer 18 is coated with a laminate like material or otherwise treated, to allow the stabilizer layer 18 to be easily removed from the attachment ring 14 with minimal force, and without damage to the attachment ring 14 or the stabilizer layer 18. An incision, or slit 20, may be may be cut through one or both opposing ends of the stabilizer layer 18 to aid in the removal of the stabilizer layer 18 without disruption to the bellows membrane 12. Positioning tabs 19b are provided at convenient positions, such as at opposing ends of the stabilizer layer 18, to further aid in the removal of the stabilizer layer 18 when desired by the user.

The release liner 22 is removably attached to the adhesive 15 to allow the device 10 to be handled without exposing the adhesive 15 to surfaces not intended for application of the device, and otherwise aid in the handling of the device 10. The release liner 22 is treated, or is comprised of a material that lends itself to easy removal from the adhesive layer 15 without damage or reducing the adhesive properties of the adhesive layer 15. In practice, the release liner 22 is removed just prior to attachment of the device 10 to the area to tissue 17 to be treated.

The amount of time required for the device 10 to be applied to the tissue 17 is dependent upon the type of treatment to be used, and the anatomical location of the affected tissue. For example, utilization of the device 10 to decontaminate a wound will likely require a relatively short-term treatment protocol of 1-3 days. However, utilization of the device 10 for bacteriostatic treatment of a wound, in order to limit or prevent the bacterial count in a wound, may require a longer term of approximately 5-7 days of treatment. Other anticipated uses of the device 10, including gene therapy or epithelial cell seeding, may require treatment durations in excess of 7 days.

Referring back to FIGS. 1 and 2, the device 10 also includes a resealable access port 24 for introduction or extraction of fluid material within the bellows membrane 12. The access port 24 is formed from an elastic, latex-like material that is capable of maintaining a fluid tight seal despite repeated penetrations, such as by a hypodermic needle 28 shown in FIG. 5, utilized to introduce material within membrane 12 of the device 10. An exemplary material for use of the access port 24 may include isoprene.

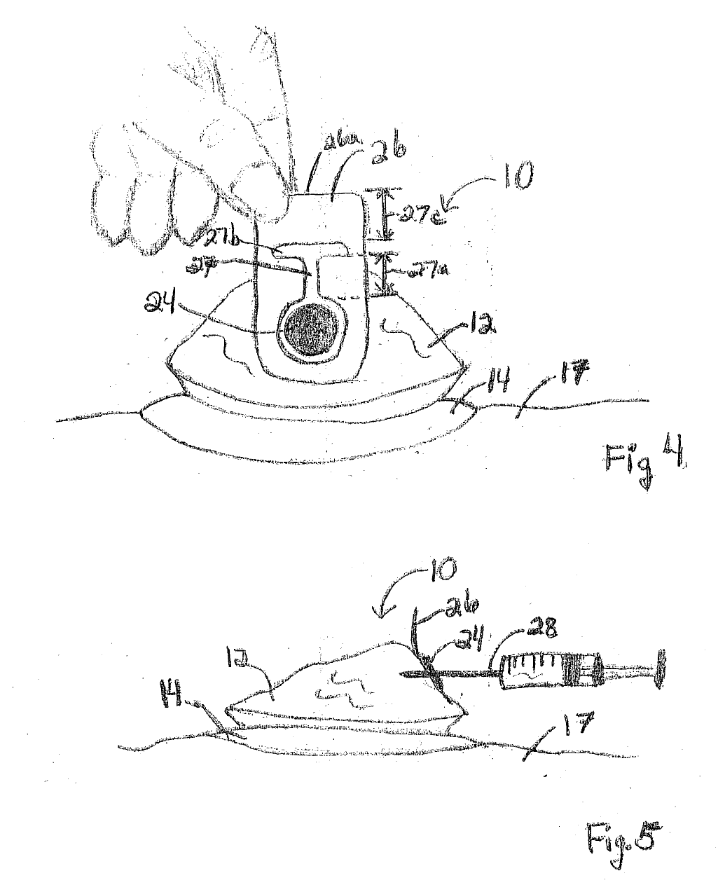

The access port 24 is preferably bonded to a finger tab 26, which in turn is bonded to the membrane 12, preferably positioned 0.5-2.0 centimeters from the outer bond 13a of the device 10. The finger tab 26, as illustrated in FIGS. 4 and 5, provides a means for lifting the access port 24 away from the tissue 17 in order to reduce the risk of injury to the patient during insertion of the needle 28. The finger tab 26 is of a size ideally suited to be easily grasped between the thumb and forefinger of a user, as depicted in FIG. 4. A reinforcing weld 27, which may be “T” shaped, is provided to prevent the access port 24 from being lifted to an angle that may risk puncturing the membrane 12 during insertion of a needle 28 through the access port 24. The distance 27a between the access port 24 and distal portion 27b of the reinforcing weld 27 is of a measurement sufficient enough to prevent the access port 24 from being positioned at an angle that allows the access port 24 to touch the inside portion of the membrane 12, or otherwise risk puncture of the membrane 12 during insertion of a needle 28, and is preferably between about 1.0-2.0 centimeters. The reinforcing weld 27 also serves to reduce the risk of injury to the user when inserting a needle 28 through the access port 24. The risk of injury is reduced by preventing the user from grasping or holding the finger tab 26 less than about 1.0-2.0 centimeters from the access port 24.

The distance 27c between the distal portion 27b of the reinforcing weld and the distal edge 26a of the finger tab 26 is of a measurement sufficient enough to allow an average sized thumb and forefinger to comfortably grasp and hold the finger tab 26, and yet not long enough to allow the user to grasp the finger tab 26 without lifting the access port 24 to a sufficient distance away from the tissue 17. The distance 27c is preferably between about 1.5-2.5 centimeters.

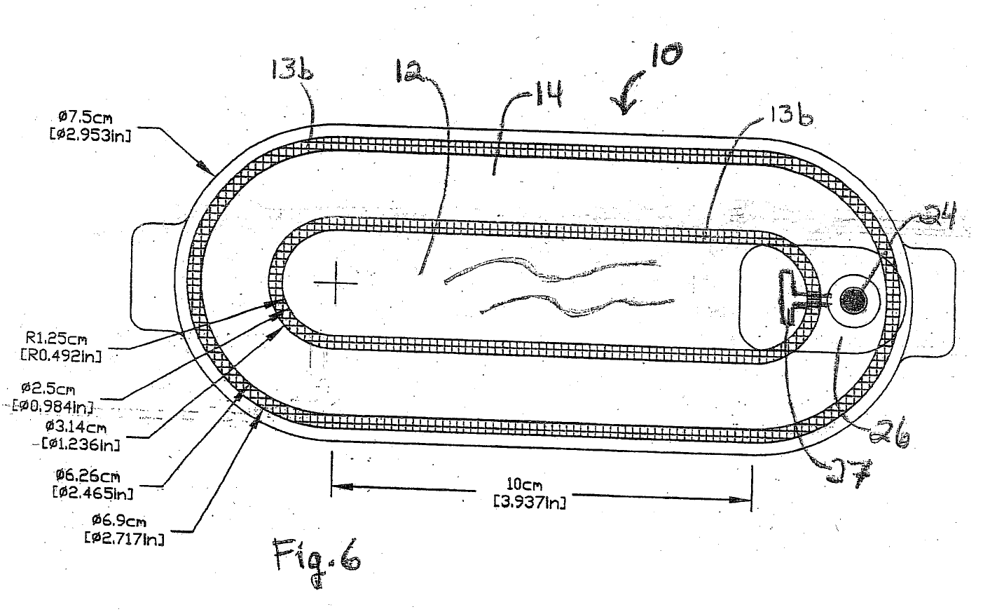

Turning now to FIG. 6, there is illustrated an alternative embodiment of the present invention, in which the device 10 is in an elongated, or elliptical form. It is anticipated that the preferred embodiment of the present invention will be a circular shape, as depicted in FIG. 1, and having an approximate diameter of about 10-14 centimeters. However, it is to be understood that the actual size and shape of the device 10 is only to be limited by the nearly infinite size and shape of wounds or tissue areas to be treated by the present invention.

While the invention has been described herein with reference to certain preferred embodiments, these embodiments have been presented by way of example only, and not to limit the scope of the invention. Accordingly, the scope of the invention should be identified only in accordance with the claims that will follow.

We claim:

1. An environmental control device for treatment of tissue, comprising:
a flexible, conformable chamber having a bellows configuration and sealable about the periphery of a wound such that the chamber portion does not adhere to the tissue;
a portal means for introduction of treatment fluids into the chamber and for extraction of fluid from said chamber; and
a lifting means for lifting said portal means away from said chamber such that introduction and extraction of fluids occurs on a plane substantially parallel to the tissue.

2. The device of claim 1 further comprising projecting means for projecting said lifting means a distance from said portal.

3. The device of claim 2 wherein said projecting means is comprised of a bonding surface for bonding a portion of said lifting means a distance from said portal.

4. The device of claim 1 wherein said portal is comprised of an elastic material, such that said portal maintains a fluid tight seal after puncture of said portal.

5. The device of claim 1 wherein a periphery portion of said chamber is coated with an adhesive material to form a fluid impermeable seal with the tissue.

6. The device of claim 1 further comprising a support layer removably adhered to said chamber.

7. The device of claim 5 wherein said support layer includes at least one non-adhesive coated projection for facilitating removal of said support layer.

8. A dressing for controlling the environment of a region of tissue, comprising:
a chamber for containment of fluids;
a portal for insertion and extraction of fluids into said chamber;
a means for adhering said chamber to tissue such that fluid contained in said chamber is in communication with the tissue; and
a means for positioning said portal away from the tissue, such that insertion and extraction of fluids occurs on a plane substantially parallel to the tissue.

9. The dressing of claim 8 wherein said portal is comprised of an elastic membrane, such that a fluid tight seal is maintained after puncture of said portal.

10. The dressing of claim 8 wherein said means for adhering said chamber to tissue is comprised of an adhesive coated contact surface fixedly attached to the periphery of said chamber for removable adhesion of dressing to tissue surrounding a wound.

11. The dressing of claim 8 wherein said chamber if formed in a bellows configuration.

12. A portal for removal and extraction of fluids to and from a tissue region, comprising:
a lifting means for lifting said portal means away from said tissue region such that introduction and extraction of fluids occurs on a plane substantially parallel to the tissue; and
an adhesive means for adhering said portal to a dressing.

13. The portal of claim 12 wherein said portal is comprised of a neoprene material.

14. A method of treating tissue comprising the steps of:
adhering a containment chamber to tissue surrounding a region of tissue to be treated;
containing a fluid treatment over the region of tissue to be treated;
providing a portal for insertion and extraction of said fluid treatment within said chamber; and
lifting said portal to a plane substantially parallel to the region of tissue to be treated during insertion and extraction of said fluid treatment.

15. The method of claim 14 further comprising the step of positioning a lift-tab a distance from said portal, such that a proximal end of said lift tab is adhered to at least a portion of said portal, and that a distal end of said lift tab is non-adhered, such that a user's thumb and forefinger are capable of grasping a top portion and bottom portion of said lift-tab, in order to lift said portal to a position substantially parallel to said region of tissue to be treated.

16. The method of claim 15 wherein said fluid treatment is comprised of antibacterial treatment of a wound within said tissue region.

17. The method of claim 15 wherein said fluid treatment is comprised of bacteriostatic treatment of a wound within said tissue region.

18. The method of claim 15 wherein said fluid treatment is comprised of continuous application stem-cells to said tissue region.

19. The method of claim 15 wherein said fluid treatment is comprised of continuous application of gene therapy drugs.

20. The method of claim 15 wherein said fluid treatment is comprised of negative pressure therapy.

21. The method of claim 15 wherein said fluid treatment is comprised of a cellular matrix gel suspension.
